SEQUENCE LISTING (2 sequences)

SEQ ID NO: 1 - DNA, 72 bp, Artificial Sequence
  Synthetic
acagcaccacagaccacccgcggatgccggtccctacgcgtcgctgtcacgctggctgtttgtcttcctgcc

SEQ ID NO: 2 - DNA, 48 bp, Artificial Sequence
  Synthetic
gggtttgggttgggttgggtttttgggtttgggttgggttgggaaaaa

(19) United States
(12) Patent Application Publication
(10) Pub. No.: US 2023/0290503 A1
(43) Pub. Date: Sep. 14, 2023

(54) METHOD OF DIAGNOSING A BIOLOGICAL ENTITY, AND DIAGNOSTIC DEVICE

(71) Applicant: OXFORD UNIVERSITY INNOVATION LIMITED, Oxford (GB)

(72) Inventors: Nicole ROBB, Oxford (GB); Nicolas SHIAELIS, Oxford (GB)

(21) Appl. No.: 17/921,417

(22) PCT Filed: Apr. 23, 2021
(86) PCT No.: PCT/GB2021/050990

§ 371 Date: Oct. 26, 2022

(30) Foreign Application Priority Data
Apr. 27, 2020 (GB) 2006144.6

(51) Int. Cl. G16H 50/20; G16H 30/40; G06T 7/00

(52) U.S. Cl. CPC G16H 50/20; G16H 30/40; G06T 7/0012; G06T 2207/20081

(57) ABSTRACT

Methods of diagnosing a biological entity in a sample are disclosed. In one arrangement image data representing one or more images of a sample is received. Each image contains plural instances of a biological entity. Each of at least a subset of the instances have at least one optically detectable label attached to the instance. The image data is preprocessed to obtain preprocessed image data. The preprocessed image data is used in a trained machine learning system to diagnose the biological entity.

### CROSS-REFERENCE TO RELATED APPLICATIONS

This application is the U.S. National Stage of International Application No. PCT/GB2021/050990, filed Apr. 23, 2021, which claims the priority benefit of the earlier filing date of GB Application No. 2006144.6, filed Apr. 27, 2020, both of which are hereby specifically incorporated herein by reference in their entirety.

### REFERENCE TO AN ELECTRONIC SEQUENCE LISTING

The contents of the electronic sequence listing (“IMIP-0100US_ST25.txt”; Size is 807 bytes and it was created on May 22, 2023, is herein incorporated by reference in its entirety.

### TECHNICAL FIELD

The present disclosure relates to diagnosing biological entities such as viruses rapidly and with high sensitivity and specificity.

### BACKGROUND OF THE INVENTION

An outbreak of the novel coronavirus SARS-CoV-2, the causative agent of COVID-19 respiratory disease, has infected millions of people since the end of 2019, resulting in many deaths and worldwide social and economic disruption. Accurate diagnosis of the virus is fundamental to response efforts.

Methods for viral diagnostics tend to be either fast and cheap at the expense of specificity or sensitivity, or vice versa. Viral culture in mammalian cells, confirmed by antibody staining, is widely quoted as the traditional “gold standard” for viral diagnosis. This approach is unsuitable, however, for point of care (POC) diagnosis because it takes several days to provide a result. Various rapid diagnostic tests based on antigen-detecting immunoassays exist for influenza and respiratory syncytial virus RSV are available, but these generally have low sensitivities compared to other methods, meaning that false negative results are common. Routine confirmation of cases of COVID-19 is currently based on detection of unique sequences of virus RNA by nucleic acid amplification tests such as real-time reverse-transcription polymerase chain reaction (RT-PCR), a process that takes a minimum of three hours.

### SUMMARY OF THE INVENTION

It is an object of the invention to provide an alternative diagnostic approach that is rapid and achieves high sensitivity and specificity.

According to an aspect of the invention, there is provided a computer-implemented method of diagnosing a biological entity in a sample, comprising: receiving image data representing one or more images of a sample, each image containing plural instances of a biological entity, each of at least a subset of the instances having at least one optically detectable label attached to the instance; preprocessing the image data to obtain preprocessed image data; and using the preprocessed image data in a trained machine learning system to diagnose the biological entity.

This methodology is demonstrated by the inventors to distinguish reliably between microscopy images of coronaviruses and two other common respiratory pathogens, influenza and respiratory syncytial virus. The method can be completed in minutes, with a validation accuracy of 90% for the detection and correct classification of individual virus particles, and sensitivities and specificities of over 90%. The method is shown to provide a superior alternative to traditional viral diagnostic methods, and thus has the potential for significant impact.

The received image data is preprocessed to obtain preprocessed image data. The preprocessed image data is used by the machine learning system to diagnose the biological entity in the sample. The preprocessing may comprise generating a plurality of sub-images for each image of the sample, each sub-image representing a different portion of the image and containing a different one of the instances of the biological entity. The sub-images may be generated such that each sub-image contains plural optically detectable labels that are colocalized, colocalization being defined as where locations of plural optically detectable labels are consistent with the optically detectable labels being attached to a same one of the instances of the biological entity (e.g. being closer to each other than a predetermined threshold related to the size of the biological entity). The generation of the sub-images may thus comprise: identifying regions where, in each region, plural optically detectable labels are colocalized, and generating a separate sub-image for each of at least a subset of the identified regions, each generated sub-image containing a different one of the identified regions. The preprocessing can therefore distinguish accurately between objects that are highly likely to correspond to instances of the biological entity (e.g. virus particles) and other objects that are less likely to correspond to instances of the biological entity (e.g. optically detectable labels that are not bound to any instance of the biological entity, which are unlikely to be located as close to each other by chance alone).

In an embodiment, the colocalized optically detectable labels (likely to be bound to the same instance of a biological entity) comprise at least two colocalized optically detectable labels of different type. The labels can therefore be distinguished from each other more easily, even when there is a high degree of overlap (such that they would otherwise be confused with a single label). This approach has been shown by the inventors to be particularly efficient where the optically detectable labels of different type comprise optically detectable labels having different emission spectra (e.g. different colours, such as green and red).

In an embodiment, the generation of the sub-images comprises using relative intensities from the colocalized optically detectable labels of different type to select a subset of the identified regions, for the generation of the sub-images, that have a higher probability of containing one and only one instance of the biological instance. This feature helps to deal with random colocalization (where optically detectable labels of different type are colocalized for reasons other than being attached to the same instance of the biological entity, for example due to aggregation of the optically detectable labels or sticky patches on a transparent substrate used for immobilization during capture of the images of the sample). The colocalized optically detectable labels of different type may be configured to have different labelling efficiency with respect to each other for the biological entity of interest, such that a ratio of intensities from the different labels is expected to be within a range of values. If a ratio of intensities from the different labels is outside of the expected range of values it is likely that the optically detectable labels are not colocalized on the biological entity.

In an embodiment, the generation of the sub-images comprises using detected axial ratios of objects in the identified regions to select a subset of the identified regions, for the generation of the sub-images, that have a higher probability of containing one and only one instance of the biological instance. Thus, knowledge of the shape of the biological entity can be used to filter out sub-image candidates that are less likely to contain the biological entity. For example, where a biological entity is known to be filamentary, sub-images containing spherical objects will be less likely to contain an instance of the biological entity and vice versa.

In an embodiment, the method further comprises detecting one or more axial ratios of objects in the generated sub-images and using the detected one or more axial ratios to select a trained machine learning system to use to diagnose the biological entity. Thus, the detection of average axial ratios may be used to select a machine learning system that is particularly appropriate for the biological entity (e.g. a machine learning system that is specifically configured and/or trained for biological entities having similar axial ratios).

In an embodiment, each sub-image is defined by a bounding box surrounding the sub-image. The bounding boxes may be defined so as to only surround groups of pixels representing objects that have an area within a predetermined size range. Thus, an area filter may be applied to objects in the image. The predetermined size range may have an upper limit and/or a lower limit. This approach allows objects having sizes that are inconsistent with being a labelled instance of the biological entity of interest to be efficiently excluded, thereby improving the quality of the data that is supplied to the machine learning system.

In an alternative aspect of the invention, there is provided a method of training a machine learning system for diagnosing a biological entity in a sample, comprising: receiving training data containing representations of one or more images of each of one or more samples and diagnosis information about a diagnosed biological entity in each sample, each image containing plural instances of the diagnosed biological entity of the corresponding sample, and each of at least a subset of the instances having at least one optically detectable label attached to the instance; and training the machine learning algorithm using the received training data.

In an alternative aspect of the invention, there is a diagnostic device, comprising: a sample receiving unit configured to receive a sample; a sample processing unit configured to cause attachment of at least one optically detectable label to at least a subset of instances of a biological entity present in the sample; a sensing unit configured to capture one or more images of the sample containing the optically detectable labels to obtain image data; and a data processing unit configured to: preprocess the image data to obtain preprocessed image data, and use the preprocessed image data in a trained machine learning system to diagnose the biological entity; or send the obtained image data to a remote data processing unit configured to preprocess the image data to obtain preprocessed image data, and use the preprocessed image data in a trained machine learning system to diagnose the biological entity.

### BRIEF DESCRIPTION OF THE DRAWINGS

Embodiments of the disclosure will be further described by way of example only with reference to the accompanying drawings, in which:

FIG. 1 is a flow chart showing a method of diagnosing a biological entity;

FIG. 2 is a schematic of an example virus labelling strategy in which positively charged calcium ions bridge a lipid membrane of a virus and negatively charged phosphate groups on an ssDNA, binding two types of fluorescently labelled ssDNA (one with a green label and one with a red label) to the surface of the virus;

FIG. 3 depicts immobilization of labelled viruses on a chitosan-coated glass slide and illumination with red and green laser light on a widefield total internal reflection fluorescence microscopy (TIRF) microscope;

FIG. 4 depicts representative field of views (FOVs), each representing an image of a sample, of fluorescently labelled CoV (IBV); the virus sample was immobilized and labelled with 0.45M CaCl₂, 1 nM Cy3 (green) DNA and 1 nM Atto647N (red) DNA before being imaged; green DNA was observed in the green channel (532 nm, top panels) and red DNA in the red channel (640 nm, middle panels); merged red and green localizations are shown in the lower panels; scale bar represents 10 μm; negative controls where virus was replaced with minimal essential media (MEM), and where CaCl₂ or DNA were replaced with water, were included;

FIG. 5 depicts a magnified image of the bottom left panel of FIG. 4 showing colocalizations of green and red DNA that correspond to doubly labelled coronavirus particles; white boxes represent examples of colocalized particles; the scale bar represents 5 μm;

FIG. 6 depicts a magnified image of the bottom panel of the second column of FIG. 4 (corresponding to FIG. 5 except that the virus is replaced with MEM); the scale bar represents 5 μm;

FIG. 7 schematically depicts a segmentation process, with (i) showing a single raw FOV (cropped for magnification), (ii) showing intensity filtering applied to i) to produce a binary image, (iii) showing area filtering applied to ii) to include only the objects with areas between 10-100 pixels, thus excluding free ssDNA and aggregates, (iv) showing the location image associated with i), (v) showing colocalized signals in the location image, (vi) showing bounding boxes (BBXs) found from iii) drawn onto v), with objects that do not meet the colocalization condition being rejected, (vii) showing bounding boxes of objects that do meet the colocalization condition drawn over i); the scale bar represents 10 μm;

FIG. 8 is a plot showing the mean number of bounding boxes per FOV for labelled CoV (IBV) and the negative controls;

FIG. 9 depicts representative FOVs of fluorescently labelled CoV (IBV), influenza (PR8 and WSN) and RSV; the virus samples were immobilized and labelled with 0.45M CaCl₂, 1 nM Cy3 (green) DNA and 1 nM Atto647N (red) DNA before being imaged; FOVs from the red channel are shown; the scale bar represents 10 μm;

FIG. 10 depicts representative FOVs of fluorescently labelled coronavirus (CoV (IBV)), two strains of H1N1 influenza (A/WSN/33 and A/PR8/8/34), RSV (strain A2) and a negative control where virus was substituted with minimal essential media (MEM); the virus sample was immobilized and labelled with 0.65M CaCl₂, 1 nM Cy3 (green) DNA and 1 nM Atto647N (red) DNA before being imaged; merged red and green localizations are shown, examples of colocalizations are highlighted with white boxes; the scale bar represents 10 μm;

FIG. 11 is a plot showing the mean number of bounding boxes per FOV for labelled CoV (IBV), influenza (PR8 and WSN) and RSV;

FIGS. 12-14 depict normalized frequency plots of the maximum pixel intensity, area, and semi-major-to-semi-minor-axis-ratio within the bounding boxes for the four different viruses;

FIG. 15 schematically illustrates an example 15-layer shallow convolutional neural network; following the input layer (bounding boxes from the segmentation process), the network consists of three convolution-ReLU layers, each followed by a batch normalisation layer (not shown in this figure) and a max pooling layer for stages 1 and 2; the classification stage has a fully-connected layer and a softmax layer to convert the output of the previous layer to a normalised probability distribution, allowing the initial input to be classified;

FIG. 16 depicts a confusion matrix showing that the trained network could differentiate positive CoV (IBV) samples from a negative control sample that contained only ssDNA with high confidence; the diagonal elements of such a matrix represent the percentage of correctly classified signals and the off-diagonal elements the false positives and negatives (i.e. 77.8% of signals were correctly classified as CoV (IBV), whilst the remaining 22.2% were incorrectly classified as ssDNA (false negatives); 85.9% of signals were correctly classified as free ssDNA, whilst the remaining 14.1% were incorrectly classified as CoV (false positives); sensitivity values for each class are given along the bottom row (upper number is the sensitivity value, lower number is the remaining percentage), specificity values in the rightmost column and the overall validation accuracy of the model in the bottom rightmost square; 3000 bounding boxes from 3 different days of experiments (1 k BBXs per day per class) were used for each class;

FIG. 17 depicts a confusion matrix that is the same as FIG. 16 but showing that the network could differentiate between CoV (IBV) and PR8;

FIG. 18 depicts a confusion matrix showing that CoV (IBV) and PR8 can both be distinguished from the negative (−Virus); 3500 bounding boxes were used for the two virus classes and 1500 bounding boxes for the negative;

FIG. 19 schematically depicts an example diagnostic device;

FIG. 20 illustrates the format of a confusion matrix; and

FIG. 21 is a graph showing trained model robustness over 135 days.

### DETAILED DESCRIPTION OF THE EXAMPLE EMBODIMENTS

Embodiments of the disclosure relate to computer-implemented methods of diagnosing biological entities in a sample. Methods of the present disclosure are thus computer-implemented. Each step of the disclosed methods may be performed by a computer in the most general sense of the term, meaning any device capable of performing the data processing steps of the method, including dedicated digital circuits. The computer may comprise various combinations of computer hardware, including for example CPUs, RAM, SSDs, motherboards, network connections, firmware, software, and/or other elements known in the art that allow the computer hardware to perform the required computing operations. The required computing operations may be defined by one or more computer programs. The one or more computer programs may be provided in the form of media or data carriers, optionally non-transitory media, storing computer readable instructions. When the computer readable instructions are read by the computer, the computer performs the required method steps. The computer may consist of a self-contained unit, such as a general-purpose desktop computer, laptop, tablet, mobile telephone, or other smart device. Alternatively, the computer may consist of a distributed computing system having plural different computers connected to each other via a network such as the internet or an intranet.

The disclosed methods are particularly applicable where the biological entity is a virus, for example a human or animal virus (i.e. a virus known to infect a human or animal). In this case the diagnosis of the virus comprises determining the identity of the virus, including for example distinguishing between one type of virus and another type of virus (e.g. to distinguish between viruses from different families). The disclosed methods may also be applied to other types of biological entity, such as bacteria. The diagnosis of the biological entity can be used as part of a method of testing for the presence or absence of a target biological entity. When the biological entity is successfully diagnosed as the target biological entity, the test has thus successfully detected the presence of the target biological entity. When the biological entity is diagnosed as a biological entity that is not the target biological entity or no diagnosis at all is obtained, the test has successfully detected the absence of the target biological entity.

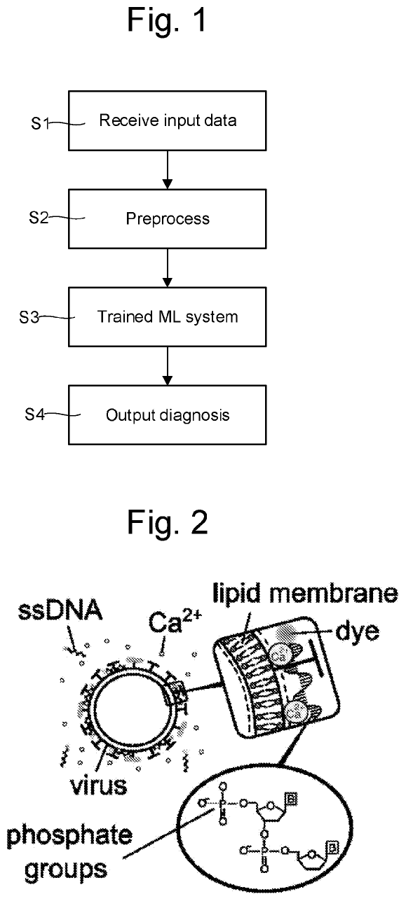

FIG. 1 is a flow chart showing a schematic framework for methods of the disclosure.

In step S1, image data is received. The image data represents one or more images of a sample. The sample contains plural instances (e.g. individual particles) of a biological entity to be diagnosed. The sample may be derived from a human or animal patient and take any suitable form (e.g. biopsy, nasal swab, throat swab, lung or bronchoalviolar fluid, blood sample, etc.). Each of at least a subset of the instances of the biological entity have at least one optically detectable label attached to them. The optically detectable labels may, for example, comprise a fluorescent or chemiluminescent label. The optically detectable labels are visible in the one or more images of the sample. However, in the absence of further steps it would be difficult to determine which of the visible labels is attached to a biological entity and which are freely floated in the sample. Furthermore, it would be difficult to reliably distinguish between different types of biological entity from visual inspection of the images. Methods of the present disclosure described below address these difficulties.

The optically detectable labelling of the instances of the biological entity can be performed in various ways, including by using antibodies, functionalised nanoparticles, aptamers and/or genome hybridisation probes for example. An efficient approach, particularly where the biological entity is an enveloped virus, is to use fluorescent labels comprising nucleic acids (e.g. DNAs or RNAs) with added fluorophores. An example of such an approach is described in detail in Robb, N. C. et al. Rapid functionalisation and detection of viruses via a novel Ca²⁺-mediated virus-DNA interaction, Sci Rep. 2019 Nov. 7; 9 (1):16219. doi: 10.1038/s41598-019-52759-5. This method uses polyvalent cations, like calcium, to bind short DNAs of any sequence to intact virus particles. It is thought that the Ca²⁺ ions derived from calcium chloride facilitate an interaction between the negatively charged polar heads of the viral lipid membrane and the negatively charged phosphates of the nucleic acid, as depicted schematically in FIG. 2. Methods of the present disclosure may preferably use this approach.

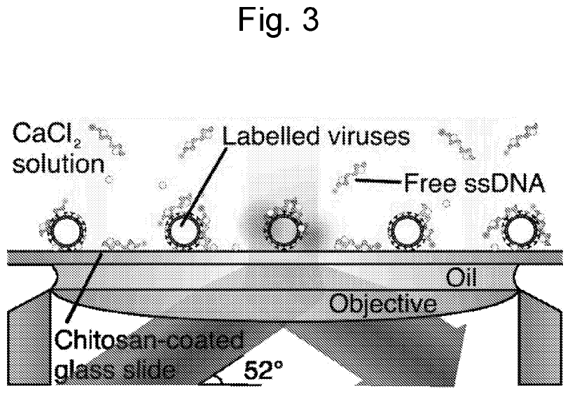

As exemplified in FIG. 3, in some embodiments the images of the sample may be obtained by immobilizing the instances of the biological entity in the sample (e.g. the fluorescently labelled viruses) on a surface of a transparent substrate (e.g. a glass slide) and imaging the biological entities (e.g. viruses) through the transparent substrate. In an embodiment, as exemplified in FIG. 3, the imaging is performed using total internal reflection fluorescence (TIRF) microscopy. The images may, however, be obtained using other microscopy methods.

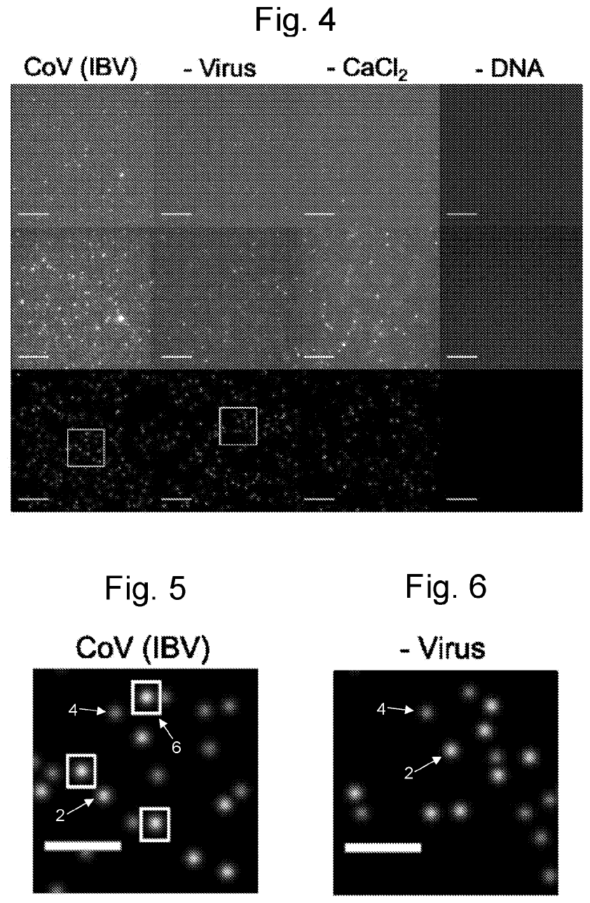

FIGS. 4-6 depict example images (which may also be referred to as field of views, FOVs) obtained using the approach of FIG. 3. In this case, a sample containing infectious bronchitis virus (IBV), an avian coronavirus (CoV), was immobilized on a substrate and labelled with 0.45M CaCl₂, 1 nM Cy3 (green) DNA and 1 nM Atto647N (red) DNA before being imaged. FIG. 4 contains a grid of 12 panels. The scale bar in each panel represents 10 μm. The top panels contain images in a red channel (i.e. images in which the red fluorescent labels contribute to the image but the green fluorescent labels do not). The middle panels contain images in a green channel (i.e. images in which the green fluorescent labels contribute to the image but the red fluorescent labels do not). The bottom panels show merged localizations for each of the red and green channels. Green spots represent locations of green labels, red spots represent locations of red labels, and yellow spots represent locations where green and red labels are simultaneously present (i.e. colocalized). The first column shows images in which the sample contained the virus, the CaCl₂ and the DNA. The second, third and fourth columns show images from negative control experiments in which, respectively: 1) the virus was replaced with minimal essential media (MEM); CaCl₂ was replaced with water; and 3) DNA was replaced with water. FIG. 5 is a magnified view of the inset box in the bottom panel of the first column. FIG. 6 is a magnified view of the inset box in the bottom panel of the second column. The scale bar in FIGS. 5 and 6 represents 5 μm.

In FIGS. 5 and 6, three types of dominant visible points (referred to herein as localizations) are seen: single isolated green localizations 2 (identifying the location of green labels); single isolated red localizations 4 (identifying the locations of red labels); and points (encircled by boxes) where a green label and a red label are located close enough together to be consistent with being attached to the same virus particle, which are referred to herein as colocalizations 6. When the virus and/or calcium chloride were omitted from the sample only single green or red localizations were observed (see FIGS. 4 and 6), while omission of the DNAs resulted in complete loss of the fluorescent signal (see FIG. 4, fourth column). It can therefore be concluded that the single localizations 2 and 4 arose from free DNA, while colocalizations 6 are caused by doubly labelled coronavirus particles. This effect may be used in a preprocessing procedure as described below.

In the framework of FIG. 1, the method further comprises preprocessing the image data in step S2 to obtain preprocessed image data. The preprocessed image data is then provided to a machine learning system which diagnoses the biological entity (in step S3) using the preprocessed image data. The diagnosis may be output in step S4 in a user interpretable form (e.g. on a display or as a data output).

In some embodiments, the preprocessing comprises generating a plurality of sub-images for each of one or more of the images of the sample that are available. Each sub-image comprises a different portion of an image represented by the image data and contains a different one of the instances of the biological entity. Each sub-image may be generated (e.g. sized and located) to contain one and only one of the instances. Thus, each sub-image may be generated so that it contains its own distinct virus particle. The generation of the sub-images may thus comprise identifying the location of each of a plurality of the instances of the biological entity in the image. The sub-images may be generated such that each sub-image contains the locations of plural optically detectable labels, and the locations of the plural optically detectable labels are consistent (e.g. close enough together) with the optically detectable labels being attached to a same one of the instances of the biological entity. Plural optically detectable labels that are located in a manner consistent with the optically detectable labels being attached to a same one of the instances of the biological entity may be referred to herein as being colocalized. The generation of the sub-images may thus comprise identifying regions where, in each region, plural optically detectable labels are colocalized, and generating a separate sub-image for each of at least a subset of the identified regions, where each generated sub-image contains a different one of the identified regions.

The sub-images may or may not contain images of each of the plural optically detectable labels. For example, when the labels have different colours, each sub-image may contain an image of only one of the labels and the locations of the different labels may be determined by overlaying different sub-images of the same region (e.g. overlaying a sub-image from a red channel with a corresponding sub-image from a green channel or overlaying a map of locations of labels from a red channel with a corresponding map of locations of labels from a green channel). In some embodiments, the locations of the instances may be identified by finding where images of different optically detectable labels overlap with each other. Statistically, a large majority of the cases where the optically detectable labels are close enough to each other to be considered colocalized (e.g. overlapping in the image and/or closer to each other than a maximum dimension of the biological entity of interest) will correspond to situations where the labels are in fact bound to the same instance of the biological entity.

As exemplified in FIGS. 4-6 and mentioned above, the sample may be arranged to contain at least two, optionally at least three, optionally at least four, different types of optically detectable label. The different types of optically detectable label may have different emission spectra (e.g. different colours, such as red and green), which makes closely spaced labels easier to distinguish from single labels (e.g. because they can be observed separately in different channels).

In some embodiments, the generation of the sub-images comprises using relative intensities (e.g. a ratio of intensities) from the colocalized optically detectable labels of different type (e.g. different colours, such as red and green) to select a subset of the identified regions, for the generation of the sub-images, that have a higher probability of containing one and only one instance of the biological instance. This feature helps to deal with random colocalization (where optically detectable labels of different type are colocalized for reasons other than being attached to the same instance of the biological entity, for example due to aggregation of the optically detectable labels or sticky patches on a transparent substrate used for immobilization during capture of the images of the sample). DNA is known to be prone to such aggregation for example. The colocalized optically detectable labels of different type may be configured to have different labelling efficiency with respect to each other for the biological entity of interest, such that a ratio of intensities from the different labels is expected to be within a range of values. This could be achieved, for example, by forming the colocalized optically detectable labels of different type using nucleic acids of different length and/or different numbers of strands (e.g. single and double stranded DNA). If a ratio of intensities from the different labels is outside of the expected range of values it is likely that the optically detectable labels are not colocalized on the biological entity.

In an embodiment, the generation of the sub-images uses detected axial ratios of objects (where an axial ratio of an object is understood to mean a ratio between the lengths of two principle axes of an object, such as a ratio between a long axis and a short axis) in the identified regions to select a subset of the identified regions, for the generation of the sub-images, that have a higher probability of containing one and only one instance of the biological instance. Thus, knowledge of the shape of the biological entity can be used to filter out sub-image candidates that are less likely to contain the biological entity. For example, where a biological entity is known to be filamentary, sub-images containing spherical objects will be less likely to contain an instance of the biological entity and vice versa.

In an embodiment, the method further comprises detecting one or more axial ratios of objects in the generated sub-images and using the detected one or more axial ratios to select a trained machine learning system to use to diagnose the biological entity. In some embodiments, an average axial ratio is obtained and used in the selection of the trained machine learning system. Thus, the detection of axial ratios (and/or average axial ratios) may be used to select a machine learning system that is particularly appropriate for the biological entity (e.g. a machine learning system that is specifically configured and/or trained for biological entities having similar axial ratios).

In some embodiments, each sub-image is defined by a bounding box. The bounding boxes are defined so as to surround only objects that have an area within a predetermined size range (i.e. area filtering is applied). An object may be defined in this context as a group of mutually adjacent pixels having an intensity that is different from an average intensity of surrounding pixels by a predetermined amount. The predetermined size range may have either or both of a lower limit and an upper limit. Objects in the image which are too small or too large to conceivably be an instance of the biological entity of interest can thus be filtered out. In specific examples discussed in the present disclosure, the predetermined size range was 10-100 pixels, but the range will depend on the particular optical settings that have been used to obtain the images (e.g. magnification, resolution, focus, etc.).

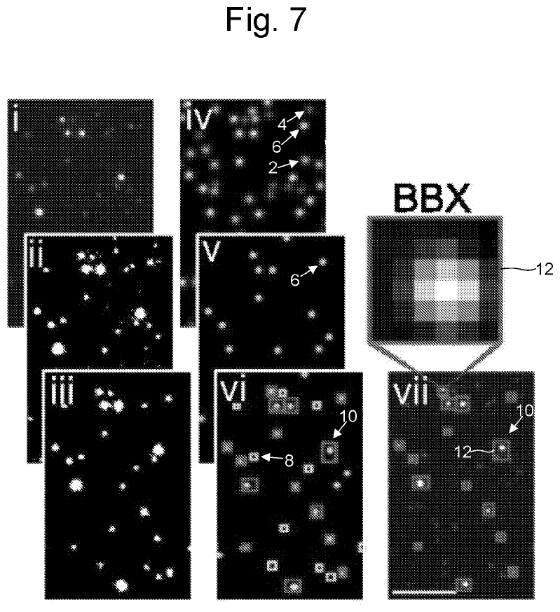

In an embodiment, the defining of the bounding boxes is performed after the image has been segmented using adaptive filtering, as exemplified in FIG. 7. Sub-figure (i) shows a single raw image (cropped for magnification). Sub-figure (ii) shows the result of intensity filtering applied to i) to produce a binary image (e.g. using MATLAB's built-in ‘imbinarize’ function). Sub-figure (iii) shows the result of area filtering applied to ii) to include only the objects with areas between 10-100 pixels, thus excluding free ssDNA and aggregates (e.g. using MATLAB's built-in ‘bwpropfilt’ function).

In an embodiment, each bounding box is defined by identifying a smallest rectangular box that contains the object to be surrounded by the bounding box and expanding the smallest rectangular box to a common bounding box size that is the same for at least a subset of the bounding boxes. Preprocessed image data can then be generated in units that all have the same size by filling a region within the bounding box outside of the smallest rectangular box with artificial padding data for each of the bounding boxes.

The preprocessing may optionally contain other steps, such as filtering the images using other expected properties of instances of the biological entities of interest. These other properties may include expected intensity ratios or axial ratios as discussed above. Alternatively or additionally, the preprocessing may include deconvolution processing to make images less dependent on detailed settings of the microscope.

The generation of the bounding boxes using the area filtering (to include only objects of a suitable size) is combined with the localization information (to include only objects where colocalized labels are present) to provide the highest quality data to the machine learning system (i.e. data units that are most easily compared with each other and with training data and which contain minimal or no units that do not correspond to instances of the biological entity that it is desired to diagnose). Later steps in this procedure are also exemplified in FIG. 7, in sub-figures (iv)-(vii). Sub-figure (iv) shows a location image associated with sub-figure i) (showing single green localizations 2, single red localizations 4 and colocalizations 6). Sub-figure (v) shows only the colocalizations 6 of the location image. Sub-figure (vi) shows bounding boxes found from iii) drawn onto v). Objects 8 that do not contain a colocalization are rejected. Sub-figure (vii) shows bounding boxes 12 of objects 10 that do meet the colocalization condition, drawn over i). The scale bar in each sub-figure represents 10 μm.

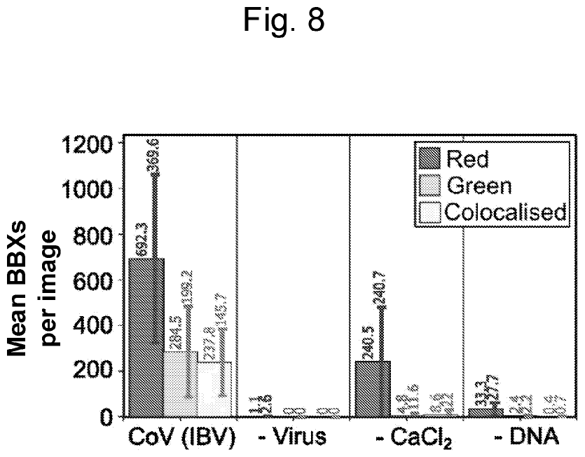

The segmentation process was fully automated, allowing each image to be processed in ˜2 seconds. FIG. 8 shows results of this analysis, confirming that the mean number of bounding boxes 12 satisfying the area filtering and containing a colocalization 6 per image (vertical axis) obtained when CoV (IBV) was present was significantly higher than when the virus, calcium chloride or DNA were omitted from the sample.

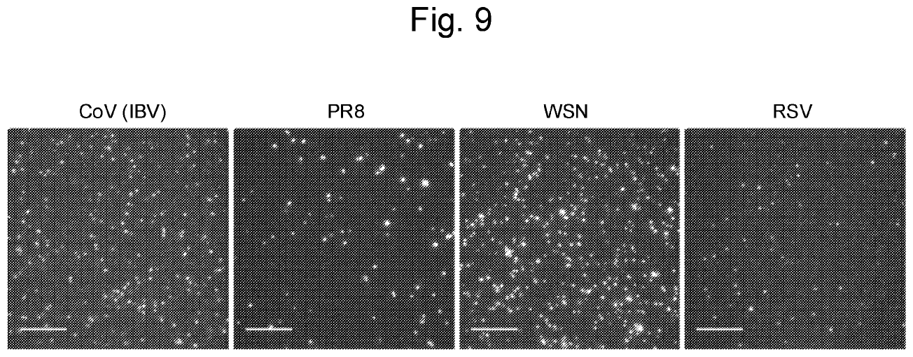

The symptoms of the early stages of COVID-19 are nonspecific, and thus diagnostic tests should preferably aim to differentiate between coronavirus and other common respiratory viruses such as influenza and respiratory syncytial virus (RSV). These viruses are similar in size and shape, and so cannot be easily distinguished from each other by eye in diffraction-limited microscope images of fluorescently labelled particles (see FIG. 9). Embodiments of the present disclosure address this problem by training a machine learning system (e.g. a neural network) to differentiate and classify images of different viruses, exemplified in detail with respect to CoV, influenza and RSV but applicable to other viruses and biological entities.

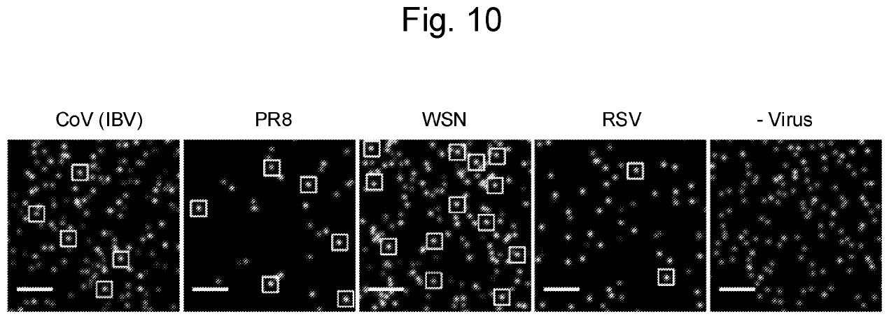

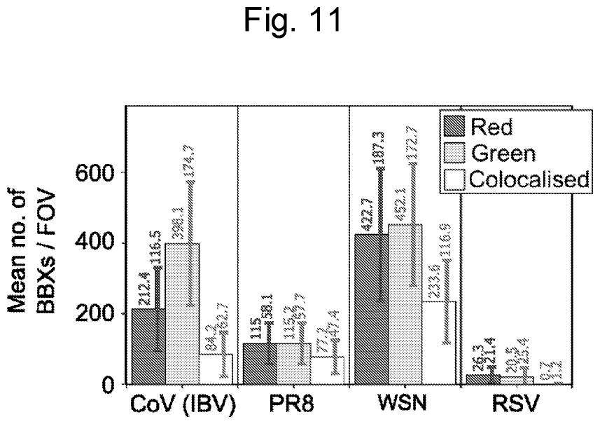

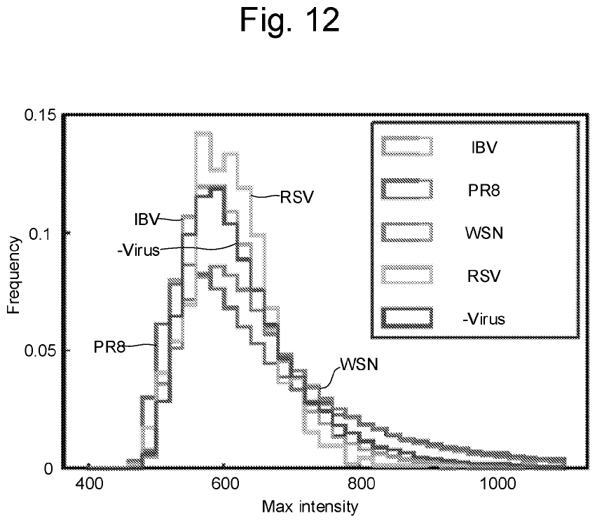

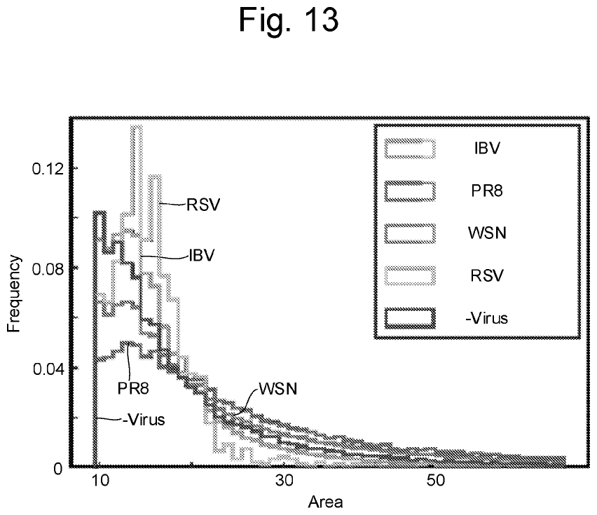

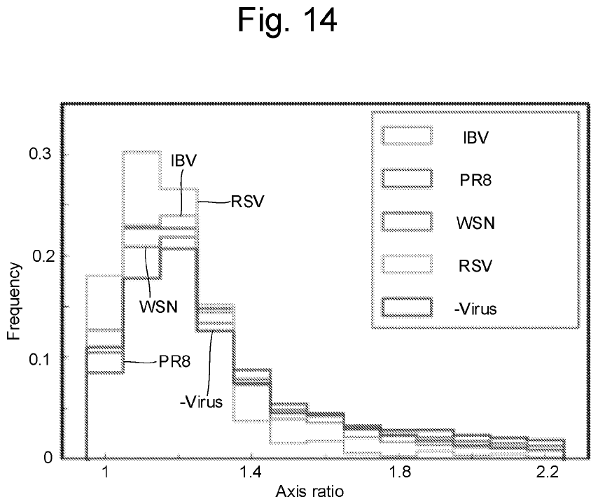

In one experiment, two H1N1 strains of influenza (A/WSN/33 and A/PR8/8/34), RSV (strain A2) and CoV (IBV) were fluorescently labelled and hundreds of field of views (FOVs) of each were acquired during an imaging step (see FIGS. 9 and 10). Movies of 5 frames per FOV (measuring 75×49 μm) were taken at 30 ms exposure. Each frame thus provides an image of a sample. To automate the task and ensure no bias in the selection of FOVs, the whole sample was scanned using the multiple acquisition capability of the microscope; 81 FOVs could be imaged in just 2 minutes. The images were then segmented as described above (see FIG. 11) and the properties of the bounding boxes were examined. The inventors expected that different types and strains of virus would have small differences in surface chemistry, size and shape, and therefore the number of fluorophores and their distribution over the surface of the viruses would differ. This was confirmed, as the four viruses exhibited differences in area, semi-major-to-semi-minor-axis-ratio and maximum pixel intensity within the bounding boxes (see FIGS. 12-14). These features, as well as other features that are not easily identifiable by the human eye, can be exploited by deep learning algorithms for classification purposes.

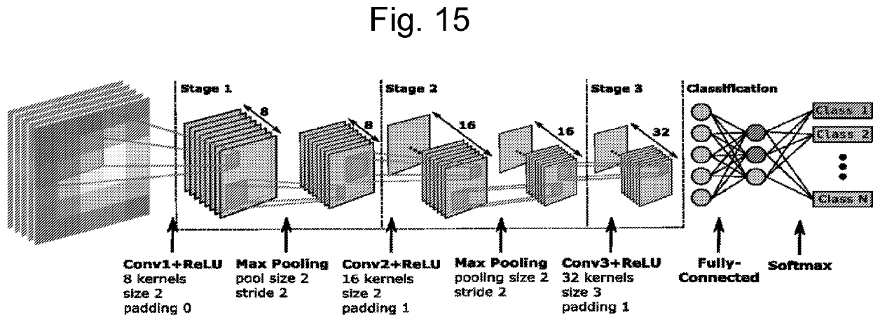

Various machine learning systems may be used. The inventors have found, however, that deep learning systems work particularly well. In one particular embodiment, the machine learning system comprises a convolutional neural network, preferably a 15-layer shallow convolutional neural network, as depicted schematically in FIG. 15. In some embodiments, different machine learning systems may be used for different levels of diagnosis. For example, a first machine learning system may be used to determine whether a sample is positive for a virus (i.e. whether any virus at all is present in the sample) and a second machine learning system may be used to diagnose the virus (if present). In the example shown, which is adapted to diagnose a virus, following on from the initial input layer (inputs comprised bounding boxes from the segmentation process), the network consisted of three stages: stages 1 and 2 each consisted of a convolution-ReLU layer to introduce non-linearity, a batch normalisation layer and a max pooling layer, while stage 3 lacked a max-pooling layer. The final classification stage had a fully-connected layer and a softmax layer for outputs.

The machine learning system may be trained in various ways. In one embodiment, training data is received by the system that contains representations of one or more images of each of one or more samples and diagnosis information about a diagnosed biological entity in each sample. Each image contains plural instances of the diagnosed biological entity of the corresponding sample. Each of at least a subset of the instances have at least one optically detectable label attached to the instance. The optically detectable labels may be attached using any of the approaches described above. The images may be obtained using any of the approaches described above. The training data may comprise image data that has been preprocessed in any of the ways described above. The machine learning system is trained using the received training data (e.g. including any preprocessing that is performed on it).

For demonstration purposes, five independent data sets of each virus strain were recorded and randomly divided into a training dataset and a validation dataset. The machine learning system (a neural network) was trained on two viruses (CoV and PR8) and a negative control containing only ssDNA and CaCl₂, using 3000 bounding boxes per strain. The data sets used for both the training and validation of the model consisted of data that was collected from three different days of experiments to ensure the validity of the method and enhance the ability of the trained models to classify data from future datasets it has never seen before. The dataset was split into the training and validation set at a ratio of 4:1. The hyperparameters remained the same throughout the training process for all models. The mini Batch size was set to 50, the maximum number of epochs to 3 and the validation frequency to 30. At the beginning of the training the first data point was at 33.3% accuracy, as expected for a completely random classification of objects into three different categories. This was followed by an initial rapid increase in validation accuracy as the network detected the more obvious parameters. As the training continued the network slowed down as the number of iterations increased further. Similarly, the Loss Function decreased accordingly. The results of the training reached 90% validation accuracies, which is comparable and in most cases superior to the sensitivity of other viral diagnostic tests.

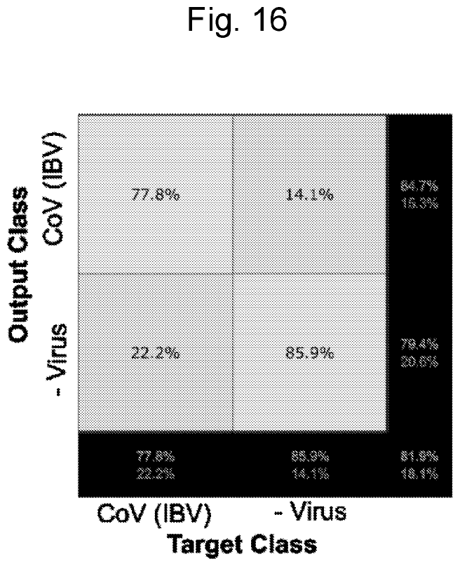

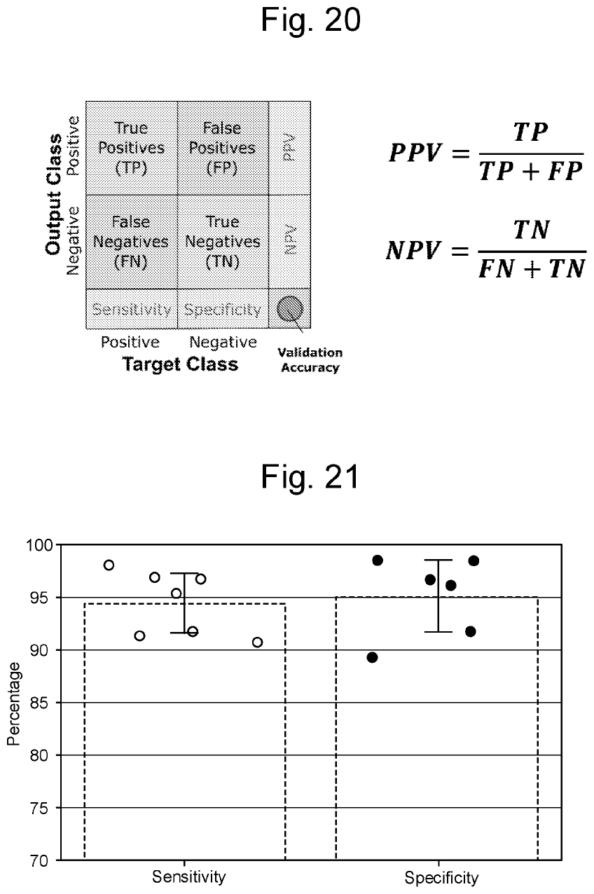

The inventors checked if the network could differentiate virus samples from non-virus samples (negative controls consisting of only calcium and DNA). The results are shown as confusion matrices in FIGS. 16-18, a common way of visualizing performance measures for classification problems. A general format of confusion matrix is depicted in FIG. 20. The rows correspond to the predicted class (Output Class), the columns to the true class (Target Class), and the far-right, bottom cell represents the overall validation accuracy of the model for each classified particle. The percentages of bounding boxes that are correctly and incorrectly predicted by the trained model are known as the positive predictive value (PPV) and negative predictive value (NPV) respectively. TP—true positive, FP—false positive, TN—true negative, FN—false negative. Thus, the diagonal elements of such a matrix represent the percentage of correctly classified viruses and the off-diagonal elements the false positives and negatives.

The trained network could differentiate positive and negative CoV (IBV) samples with high confidence (82%) (FIG. 16). This probability refers to single virus particles in the sample and not the whole sample; the probability of identifying correctly a sample with hundreds or thousands of such particles will therefore approach 100%.

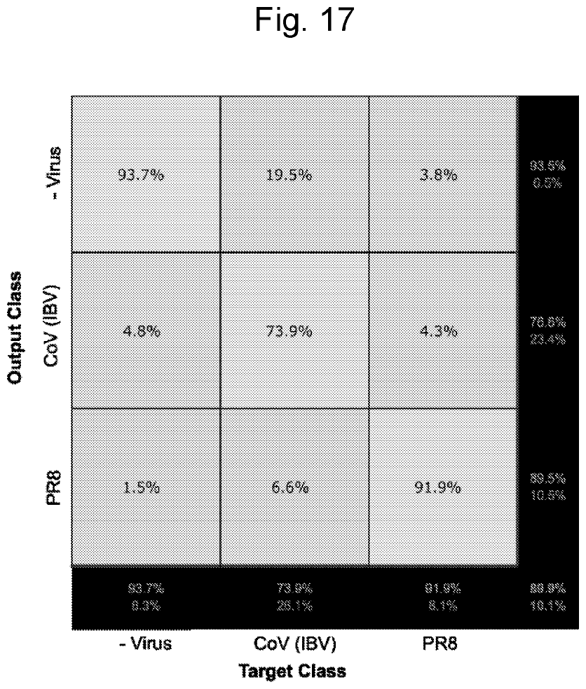

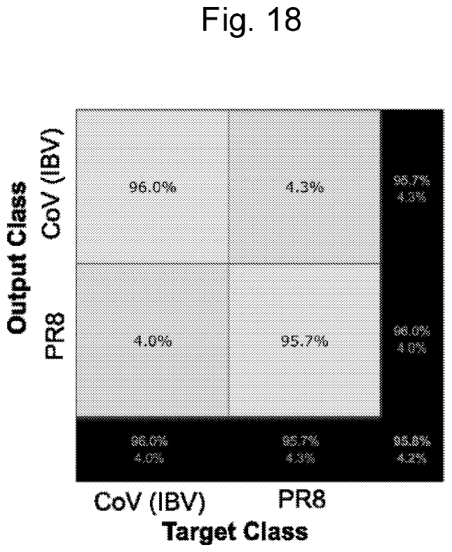

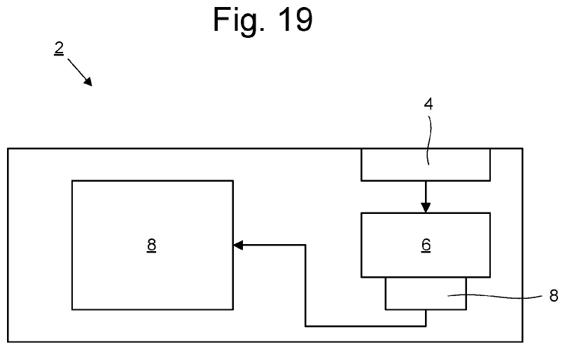

To further test the ability of the network to distinguish positives from negatives but also whether it can differentiate between viruses, the network was trained on data from the negative control, CoV (IBV) and PR8. This time an imbalanced data set was used, with a higher number of bounding boxes for the virus classes (3000 bounding boxes compared to 1,500 bounding boxes for the negative control) resulting in a model with high specificity (93.5%) and sensitivity (93.7%) towards recognizing the negative samples (see FIG. 17). This model also shows that PR8 is relatively easy to distinguish, with a sensitivity of 91.9% and specificity of 89.5%. A third model was trained (see FIG. 18), where CoV (IBV) was directly compared to PR8. The overall validation accuracy reached 95.8% with over 95% for both sensitivity and specificity per BBX for both strains. This proves that a first ‘biased’ model can be used to check whether a sample contains a virus and then a second model can be used to distinguish which specific strain or strains are present in the sample.

FIG. 21 is a graph showing trained model robustness over 135 days. Each data point (open circle for sensitivity; filled circle for specificity) corresponds to the classification result for signals detected at different dates over a period of 135 days. The network was trained on data from images of the virus IBV and allantoic fluid as a negative control. Error bars represent standard deviation.

The above demonstrates the use of fluorescence single-particle microscopy combined with deep learning to rapidly detect and classify viruses, including coronaviruses. The methods and analytical techniques developed here are applicable to the diagnosis of many pathogenic viruses. The protocols described will enable a large-scale, extremely rapid and high-throughput analysis of patient samples, yielding crucial real-time information during pandemic situations.

In an embodiment, the method is implemented by a diagnostic device 2. The diagnostic device 2 may be a standalone device or even a portable device. In an embodiment, the device 2 comprises a sample receiving unit 4. The sample receiving unit 4 is configured to receive a sample for analysis. The sample receiving unit 4 may be configured in any of the various known ways for handling samples in medical diagnostic devices (e.g. fluidics or microfluidics could be used to move the sample, immobilise, label and image it). The device 2 further comprises a sample processing unit 6 configured to cause attachment of at least one optically detectable label to at least a subset of instances of a biological entity present in the sample. The sample processing unit 6 may therefore comprise a reservoir containing suitable reagents (e.g. fluorescent labels). The device 2 further comprises a sensing unit 8 configured to capture one or more images of the sample containing the optically detectable labels to obtain image data. The device further comprises a data processing unit 8 that preprocesses the image data to obtain preprocessed image data and uses the preprocessed image data in a trained machine learning system to diagnose the biological entity. The preprocessed may be performed using any of the methods described above. The trained machine learning system may be implemented within the device 2 or the device 2 may communicate with an external server that implements the trained machine learning system. For example, the data processing unit 8 may alternatively be configured to send the obtained image data to a remote data processing unit configured to preprocess the image data to obtain preprocessed image data, and use the preprocessed image data in a trained machine learning system to diagnose the biological entity.

### Further Details about Methods

### Virus Strains and DNAs

The influenza strains (H1N1 A/WSN/1933 and A/Puerto Rico/8/1934) and RSV (A2) used in this study have been described previously in Robb, N. C. et al. Rapid functionalisation and detection of viruses via a novel Ca²⁺-mediated virus-DNA interaction, Sci Rep. 2019 Nov. 7; 9 (1):16219. doi: 10.1038/s41598-019-52759-5. Briefly, WSN, PR8 and RSV were grown in Madin-Darby bovine kidney (MDBK), Madin-Darby canine kidney (MDCK) cells and Hep-2 cells respectively. The cell culture supernatant was collected and the viruses were titred by plaque assay. Titres of WSN, PR8 and RSV were 3.3×10⁸ plaque forming units (PFU)/mL, 1.05×10⁸ PFU/mL and 1.4×105 PFU/mL respectively. The coronavirus IBV (Beaudette strain) was grown in embryonated chicken eggs and titred by plaque assay (1×10⁶ PFU/mL). Viruses were inactivated by shaking with 2% formaldehyde before use.

Single-stranded oligonucleotides labelled with either red or green dyes were purchased from IBA (Germany). The ‘red’ DNA was modified at the 5′ end with ATTO647N (5′ACAGCACCACAGACCACCCGCGGATGCCGGTCCCTACGCGTCGCTGTCACGCT GGCTGTTTGTCTTCCTGCC 3′) (SEQ ID NO: 1) and the ‘green’ DNA was modified at the 3′ end with Cy3 (5′GGGTTTGGGTTGGGTTGGGTTTTTGGGTTTGGGTTGGGTTGGGAAAAA 3′) (SEQ ID NO: 2).

### Sample Preparation

Glass slides were treated with 0.015 mg/mL chitosan (a linear polysaccharide) in 0.1 M acetic acid for 30 min before being washed thrice with MilliQ water. Unless otherwise stated, virus stocks (typically 10 μL) were diluted in 0.45 M CaCl₂ and 1 nM of each fluorescently-labelled DNA in a final volume of 20 μL, before being added to the slide surface. Negatives were taken using Minimal Essential Media (Gibco) in place of the virus. The sample was imaged using total internal reflection fluorescence microscopy (TIRF). The laser illumination was focused at a typical angle of 52° with respect to the normal. Typical acquisitions were 5 frames, taken at a frequency of 33 Hz and exposure time of 30 ms, with laser intensities kept constant at 0.78 kW/cm² for the red (640 nm) and 1.09 kW/cm² for the green (532 nm) laser.

### Instrumentation

Images were captured using wide-field imaging on a commercially available fluorescence Nanoimager microscope (Oxford Nanoimaging, https://www.oxfordni.com/), as previously described in Robb, N. C. et al. Rapid functionalisation and detection of viruses via a novel Ca²⁺-mediated virus-DNA interaction, Sci Rep. 2019 Nov. 7; 9 (1): 16219. doi: 10.1038/s41598-019-52759-5. The multiple acquisition function of the microscope was used to scan the whole sample and automate the acquisition process.

### Data Segmentation

Each raw field of view (FOV) in the red channel was turned into a binary image using MATLAB's built-in imbinarize function with adaptive filtering turned on. Adaptive filtering uses statistics about the neighbourhood of each pixel it operates on to determine whether the pixel is foreground or background. The filter sensitivity is variable-associated, with adaptive filtering which, when increased, makes it is easier to pass the foreground threshold. The bwpropfilt function was then used to exclude objects with an area outside the range 10-100 pixels, aiming to disregard free ssDNA and aggregates. The regionprops function was employed to extract properties of each found object: area, semi-major to semi-minor axis ratio (or simply, axis ratio), coordinates of the object's centre, bounding box (BBX) encasing the object, and maximum pixel intensity within the BBX.

Accompanying each FOV is a location image (LI) summarising the locations of signals received from each channel (red and green). Colocalised signals in the LI image are shown in yellow. Objects found in the red FOV were compared with their corresponding signal in the associated LI. Objects that did not arise from colocalised signals were rejected. The qualifying BBXs were then drawn onto the raw FOV and images of the encased individual viruses were saved.

Machine Learning

The bounding boxes (BBX) from the data segmentation have variable sizes but due to the size filtering they are never larger than 16 pixels in any direction. Thus, all the BBX are augmented such that they have a final size of 16×16 pixels, by means of padding (adding extra pixels with 0 grey-value until they reach the required size). The augmented images are then fed into the 15-layer CNN. The network has 3 convolutional layers in total, with kernels of 2×2 for the first two convolutions and 3×3 for the last one. The learning rate was set to 0.01 and the learning schedule rate remained constant throughout the training.

In the classification layer, trainNetwork takes the values from the softmax function and assigns each input to one of the K mutually exclusive classes using the cross entropy function for a 1-of-K coding scheme. The loss function is given by:

$$loss = {\sum }_{i = 1}^{N} {\sum }_{j = 1}^{K} {t}_{ij} ln ( {y}_{ij} )$$

where N is the number of samples, K is the number of classes, tᵢⱼ is the indicator that the i^{th} sample belongs to the j^{th} class, and yᵢⱼ is the output for sample i for class j, which in this case, is the value from the softmax function. That is, it is the probability that the network associates the i^{th} input with class j.

### Statistical Analysis

The sensitivity and specificity are common metrics for the assessment of the utility and performance of any diagnostic test. In order to understand how these are calculated we need to introduce the following terms:

True positive (TP): the patient has the disease and the test is positive,

False Positive (FP): the patient does not have the disease and the test is positive,

True negative (TN): the patient does not have the disease and the test is negative and

False negative (FN): the patient has the disease but the test is negative.

Sensitivity refers to the ability of the test to correctly identify those patients with the disease. It can be calculated by dividing the number of true positives over the total number of positives.

$$Sensitivity = \frac{TP}{TP + FN}$$

Specificity refers to the ability of the test to correctly identify those patients without the disease. It can be calculated by dividing the number of true negatives over the total number of negatives.

$$Specificity = \frac{TN}{TN + FP}$$

1. A computer-implemented method of diagnosing a biological entity in a sample, comprising:
receiving image data representing one or more images of a sample, each image containing plural instances of a biological entity, each of at least a subset of the instances having at least one optically detectable label attached to the instance;
preprocessing the image data to obtain preprocessed image data; and
using the preprocessed image data in a trained machine learning system to diagnose the biological entity.

2. The method of claim 1, wherein the preprocessing comprises generating a plurality of sub-images for each image of the sample, each sub-image representing a different portion of the image and containing a different one of the instances of the biological entity.

3. The method of claim 2, wherein the sub-images are generated such that each sub-image contains one and only one of the instances of the biological entity.

4. The method of claim 2, wherein the generation of the sub-images comprises:
identifying regions where, in each region, plural optically detectable labels are colocalized, colocalization being defined as where locations of plural optically detectable labels are consistent with the optically detectable labels being attached to a same one of the instances of the biological entity; and
generating a separate sub-image for each of at least a subset of the identified regions, each generated sub-image containing a different one of the identified regions.

5. The method of claim 4, wherein the colocalized optically detectable labels comprise at least two colocalized optically detectable labels of different type.

6. The method of claim 5, wherein the colocalized optically detectable labels of different type comprise optically detectable labels having different emission spectra.

7. The method of claim 6, wherein the generation of the sub-images comprises using relative intensities from the colocalized optically detectable labels of different type to select a subset of the identified regions, for the generation of the sub-images, that have a higher probability of containing one and only one instance of the biological instance.

8. The method of claim 7, wherein the colocalized optically detectable labels of different type are configured to have different labelling efficiency with respect to each other, preferably by forming the colocalized optically detectable labels of different type using nucleic acids of different length and/or different numbers of strands.

9. The method of claim 4, wherein the generation of the sub-images comprises using detected axial ratios of objects in the identified regions to select a subset of the identified regions, for the generation of the sub-images, that have a higher probability of containing one and only one instance of the biological instance.

10. The method of claim 2, further comprising detecting one or more axial ratios of objects in the generated sub-images and using the detected one or more axial ratios to select a trained machine learning system to use to diagnose the biological entity.

11. The method of claims 2, wherein each sub-image is defined by a bounding box surrounding the sub-image.

12. The method of claim 11, wherein the bounding boxes are defined so as to surround only objects that have an area within a predetermined size range, preferably wherein the predetermined size range has an upper limit and/or a lower limit.

13. The method of claim 10, wherein:
each bounding box is defined by identifying a smallest rectangular box that contains the object to be surrounded by the bounding box and expanding the smallest rectangular box to a common bounding box size that is the same for at least a subset of the bounding boxes; and
generation of the preprocessed image data comprises filling a region within the bounding box outside of the smallest rectangular box with artificial padding data.

14. The method of claim 1, further comprising training a machine learning system to provide the trained machine learning system, wherein the training of the machine learning system comprises:
receiving training data containing representations of one or more images of each of one or more samples and diagnosis information about a diagnosed biological entity in each sample, each image containing plural instances of the diagnosed biological entity of the corresponding sample, and each of at least a subset of the instances having at least one optically detectable label attached to the instance; and
training the machine learning algorithm using the received training data.

15. A method of training a machine learning system for diagnosing a biological entity in a sample, comprising:
receiving training data containing representations of one or more images of each of one or more samples and diagnosis information about a diagnosed biological entity in each sample, each image containing plural instances of the diagnosed biological entity of the corresponding sample, and each of at least a subset of the instances having at least one optically detectable label attached to the instance; and
training the machine learning algorithm using the received training data.

16. The method of claim 1, wherein the biological entity is a virus or bacterium.

17. The method of claim 1, wherein the machine learning system comprises a deep learning system.

18. The method of claim 1, wherein the machine learning system comprises a convolutional neural network, preferably a 15-layer shallow convolutional neural network.

19. The method of claim 1, wherein each of one or more of the optically detectable labels is a fluorescent label.

20. The method of claim 1, wherein each of one or more of the optically detectable labels is attached using any one or more of the following:
antibodies; functionalised nanoparticles; aptamers; and genome hybridisation probes.

21. The method of claim 1, wherein each of one or more of the optically detectable labels comprises a nucleic acid with an added fluorophore.

22. A computer program comprising instructions which, when the program is executed by a computer, cause the computer to carry out the method of claim 1.

23. A computer-readable medium comprising instructions which, when executed by a computer, cause the computer to carry out the method of claim 1.

24. A method of diagnosing a biological entity, comprising:
providing a sample comprising plural instances of a biological entity;
attaching at least one optically detectable label to at least a subset of the instances in the sample;
capturing one or more images of the sample containing the optically detectable labels to obtain image data; and
using the method of claim 1 to diagnose the biological entity using the obtained image data as the received image data.

25. A diagnostic device, comprising:
a sample receiving unit configured to receive a sample;
a sample processing unit configured to cause attachment of at least one optically detectable label to at least a subset of instances of a biological entity present in the sample;
a sensing unit configured to capture one or more images of the sample containing the optically detectable labels to obtain image data; and
a data processing unit configured to:
preprocess the image data to obtain preprocessed image data, and use the preprocessed image data in a trained machine learning system to diagnose the biological entity; or
send the obtained image data to a remote data processing unit configured to preprocess the image data to obtain preprocessed image data, and use the preprocessed image data in a trained machine learning system to diagnose the biological entity.
